(19) United States
(12) Patent Application Publication
(10) Pub. No.: US 2020/0105393 A1
(43) Pub. Date: Apr. 2, 2020

(54) DRUG CONTAINER LABELING METHOD AND APPARATUS

(71) Applicant: Codonics, Inc., Middleburg Heights, OH (US)

(72) Inventors: Gary Keefe, Brecksville, OH (US); Lawrence Srnka, Northfield Center, OH (US); Michael Kolberg, Hinckley, OH (US); Peter Botten, Lakewood, OH (US)

(21) Appl. No.: 16/499,824

(22) PCT Filed: Mar. 29, 2018
(86) PCT No.: PCT/US18/25115

§ 371 Date: Sep. 30, 2019

Related U.S. Application Data
(60) Provisional application No. 62/478,358, filed on Mar. 29, 2017.

(51) Int. Cl. G16H 20/17; A61M 5/315; G16H 10/65; G06K 7/14

(52) U.S. Cl. CPC G16H 20/17; A61M 5/315; G16H 10/65; G06K 7/1413; A61M 2205/3334; A61M 2005/3126; A61M 2205/50; A61M 2205/6009; A61M 2205/18; A61M 2205/3379; A61M 2205/6072

(57) ABSTRACT

Provided is a syringe reader includes a receiver comprising a releasable fastener component that cooperates with a compatible portion of the syringe to releasably couple the syringe to the syringe receiver, and maintain cooperation between the syringe and the syringe receiver during administration of the drug. A sensor is arranged adjacent to an outlet of the syringe to sense a quantity of the drug expelled from the drug container in response to exertion of a biasing force on a plunger of the syringe, and transmit a signal indicative of the sensed quantity of the drug. A barcode reader is arranged to read a barcode applied to a barrel region of the syringe installed on the syringe reader by the receiver. The barcode reader includes an optics system that emits an optical signal to interrogate the barcode.

### BACKGROUND OF THE INVENTION

### Field of the Invention

This application relates generally to a method and apparatus for labeling a drug delivery container and, more specifically, to a method and apparatus for applying an identifier onto a delivery container such as a syringe, on demand.

### 2. Description of Related Art

Industry and/or government regulations require delivery containers used to administer drugs to patients to be labeled with information that identifies those drugs. To comply with regulations and industry standards, labels on drug delivery containers must include certain information about the drug such as drug name, total dose, total volume, concentration, or other information pertaining to the drug. In addition to human-readable content labels have evolved to also include computer-readable codes such as barcodes, RFID tags, or other information encoding objects that are read by a barcode scanner, RFID reader, or other suitable device to obtain information about the drug.

Conventional labeling devices typically require at least a portion of the information encoded by a barcode on the label of a drug delivery container, for example, to be entered into the labeling device at a time when a syringe is being prepared with the drug. During drug preparation, the syringe label content is prepared and the label is printed to be ready for application to the syringe once the syringe has been prepared. However, since the labeling device produces a printed label independent of the action of physically preparing the drug into the syringe and can be applied to the syringe at any time convenient to the clinician, the syringe is available to be prepared while the label is separately published. Further, since such labels can be applied anywhere on the syringe at the discretion of the clinician, the position of the barcode or other computer-readable code provided to the labels is not predictable. As such, readers of the computer-readable codes applied to labels that can be freely-applied to syringes or other delivery containers cannot be positioned at a fixed location from where they can expect to routinely read those codes.

### BRIEF SUMMARY OF THE INVENTION

According to one aspect, the subject application involves a syringe reader for measuring a flow of a drug administered from a syringe, representing a quantity of a drug administered from the syringe. The syringe reader includes a receiver comprising a releasable fastener component that cooperates with a compatible portion of the syringe to releasably couple the syringe to the syringe reader, and maintain cooperation between the syringe and the syringe reader during administration of the drug. A sensor is arranged adjacent to an outlet of the syringe to sense a flow of the drug expelled from the drug container in response to exertion of a biasing force on a plunger of the syringe, and transmit a signal indicative of the sensed flow of the drug. A code reader is arranged to read a machine-readable code applied to a barrel region of the syringe installed on the syringe reader by the receiver, wherein the code reader comprises a system that emits an interrogation signal to interrogate the machine-readable code applied to the barrel region.

According to another aspect, the subject application involves a method of documenting administration of a drug from a syringe coupled to a syringe reader by a receiver comprising a releasable fastener. The method includes, with a sensor provided to the syringe reader, sensing a quantity of the drug expelled from the drug container in response to exertion of a biasing force on a plunger of the syringe, and transmitting a signal indicative of the sensed quantity of the drug. With a barcode reader provided to the syringe reader, a barcode applied to a barrel region of the syringe is read while the syringe is installed on the syringe reader by the receiver. Reading the barcode with the barcode reader involves emitting an optical signal from an optics system having a fixed position relative to the receiver to interrogate the barcode. The fixed position of the optics system is separated from a location where a Luer taper of the syringe installed on the syringe reader is located.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a labeling terminal for publishing labels to be applied to a barrel of a syringe or a portion of any other delivery container to identify a drug to be administered using the delivery container;

FIG. 2 schematically shows an illustrative embodiment of components included as part of the embodiment of the labeling terminal shown in FIG. 1;

FIG. 3 shows an illustrative embodiment of a drug cart and drug cart controller in communication with a labeling terminal, the labeling terminal being operatively connected to communicate with an external printer that is configured to print a barcode onto a Luer taper portion of a syringe;

FIG. 4 is a plan view of an illustrative embodiment of a syringe, with a Luer taper portion of the syringe enlarged to illustrate the barcode applied thereto;

FIG. 5 schematically shows an illustrative embodiment of components included as part of the external printer in communication with the labeling terminal shown in FIG. 3;

FIG. 6 shows an illustrative embodiment of a display in communication with a syringe reader that measures a dose of a drug actually administered to the patient from the syringe in real time, where the syringe reader includes a barcode reader positioned to read a barcode applied to a Luer taper portion of a syringe installed on the syringe reader;

FIG. 7 shows another illustrative embodiment of a display in communication with a syringe reader that measures a dose of a drug actually administered to the patient from the syringe in real time, where the syringe reader includes a barcode reader positioned to read a barcode applied to a label that extends at least partially around a circumference of a barrel region of the syringe while the syringe is installed on the syringe reader;

FIG. 8 shows a flow diagram graphically depicting a process performed by an external printer for providing a barcode onto a Luer taper of a syringe;

FIG. 9 shows a flow diagram graphically depicting a process performed by a labeling terminal to cause an external printer to apply a barcode onto a Luer taper of a syringe; and

FIG. 10 shows a flow diagram graphically depicting a process performed by a labeling terminal to produce a label for a syringe with a pre-printed barcode on a Luer taper of the syringe.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

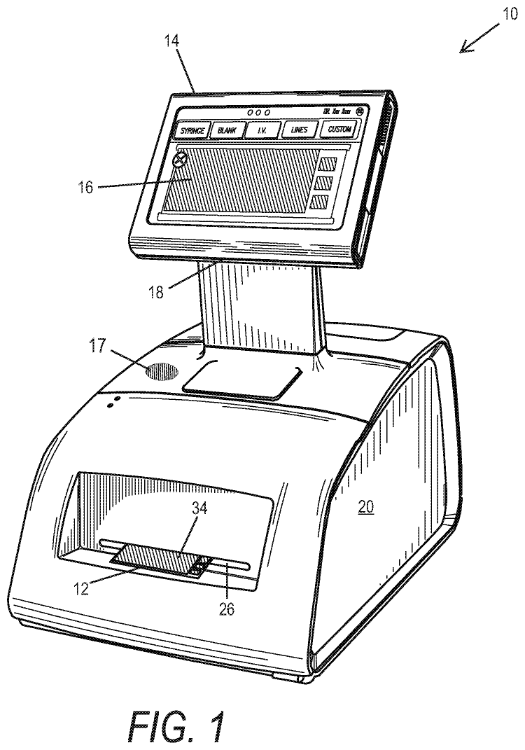

As shown in FIG. 1, the labeling terminal 10 includes a touch-screen display 14 that can be pivotally coupled to a cabinet 20 to display a virtual label 16 comprising label content 34 that will be printed onto a label 12. Once printed by a printer 26, the label 12 will be applied to extend at least partially around a circumference of a barrel region 37 of the syringe 35 to identify a drug stored in the barrel region 37 of a prepared syringe 35 to be administered to a patient.

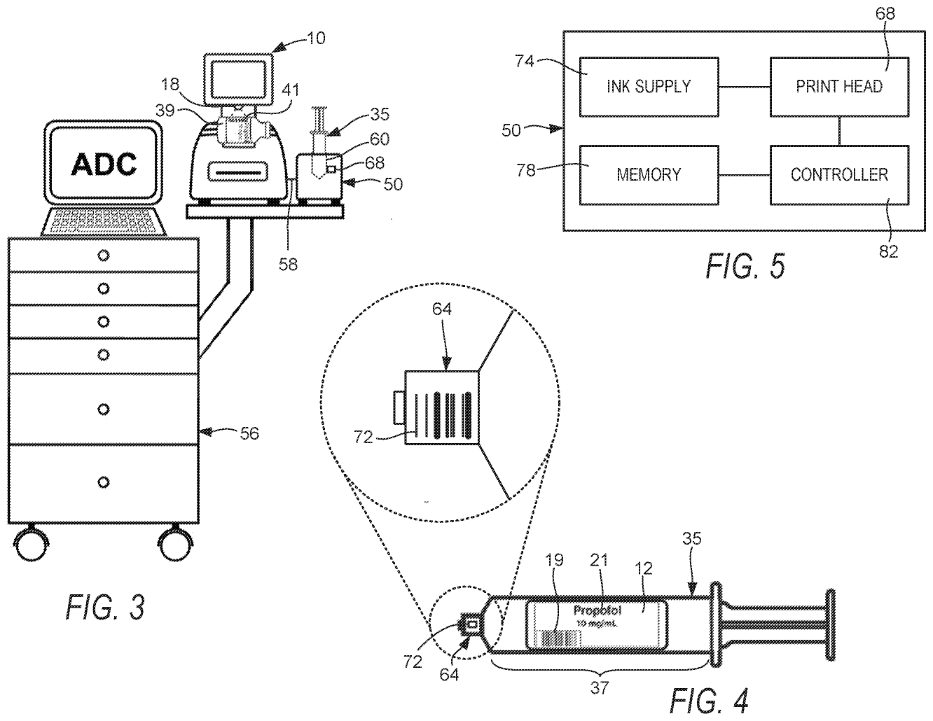

The labeling terminal 10 also includes a non-contact scanner 18 provided at a convenient location, such as integrally formed in a bottom portion of the display 14. The scanner 18 (e.g., barcode reader) reads a computer-readable code shown as a barcode 41 in FIG. 3, although other codes readable by a computer such as a RFID tag, or any other such code provided to a vial 39 supported beneath the scanner 18 are also encompassed by the present disclosure. Integrally forming the scanner 18 as part of the display 14 provides for space savings and eliminates the need to hold the scanner 18 compared to an arrangement where the scanner 18 is formed as a separate peripheral, which can be repositioned relative to the display 14. However, other embodiments can allow for a separate and distinct scanner 18 and display 14, or any other portion of a terminal that is operable to scan a computer-readable code and print the label 12 to be applied to the syringe 35 as described in U.S. Pat. No. 9,262,585 to Keefe et al., which is incorporated by reference herein in its entirety. The display 14 can display soft keys that, when touched by a clinician or any other user, inputs additional data and/or commands into the labeling terminal 10, optionally to supplement the data obtained in response to reading the computer-readable code. The virtual label 16 is a computer-generated rendering of the label 12 that offers the user a preview for visual confirmation of the appearance of the physical label 12 to be printed by the printer 26. The printer 26 prints the label 12 in compliance with a labeling standard enacted by a medical regulatory agency, professional organization, governing body, government agency. As printed, the label 12 will also include a barcode 19 (FIG. 4) encoding at least a portion of the human-readable content 21 that is printed on to the label 12. For example, the human-readable content includes, but is not limited to, alpha, numeric, or alphanumeric text instead of an arrangement of bars, squares, pixels, etc. that are required to be interpreted and translated into human-readable form understandable to a clinician by a computer.

The printer can optionally be an inkjet printer, a laser printer, or any other suitable printing device. For inkjet embodiments of the printer 26, the printer 26 can be provided with ink that is not purely black in color. It is believed that printing the barcode 19 (FIG. 4) onto a label 12 with lines or squares (for two-dimensional barcodes) that are absolute black in color, creating the highest possible contrast with a white background, results in the over-application of ink by the printer 26 onto the label stock. Application of too much ink causes the label stock to wick away the excess ink from the regions of the label stock that are being oversaturated to create the barcode 19 in black. The result is that the regions of the label stock wicking away the black ink degrades the sharpness of the lines, squares or other symbols used to create the barcode 19. Such degradation can negatively impact the readability of the resulting barcode 19, which could potentially cause the barcode to be misread by barcode scanners. In an effort to avoid this wicking effect, the printer 26 can be configured to print the barcode 19 to include lines, squares, pixels or other symbols in a color having a contrast that is at least ten (10%) percent lower than the contrast of such lines, squares, pixels or other symbols printed to be black relative to a white background. According to alternate embodiments, the lines, squares, pixels or other symbols can optionally be printed in a color having a contrast that is at least fifteen (15%) percent, or at least twenty (20%) percent lower than the contrast of such lines, squares, pixels or other symbols printed to be black relative to a white background.

The embodiment of the scanner 18 described above is a barcode reader 18, but a radio-frequency identification (“RFID”) tag reader, or any other device that reads a machine-readable code such as a two-dimensional barcode, RFID code, or any other machine-readable code without requiring contact between the labeling terminal 10 and the code be utilized as the scanner 18. According to alternate embodiments, the display 14 can be also be utilized by a user as the computer-input peripheral. For such embodiments, the soft keys displayed by the display 14 can be selected to input information such as a medicinal substance being prepared to be administered to a patient, select a correct drug identification from among a plurality of candidates returned in response to reading the barcode 41, or enter other information to be utilized in generating the label as described herein. A speaker 17 can optionally be provided to the display 14 or any other portion of the labeling terminal 10 to broadcast audible sounds for further confirmation purposes or to issue an audible alert to a clinician concerning the drug in the vial 39 labeled with the barcode 41.

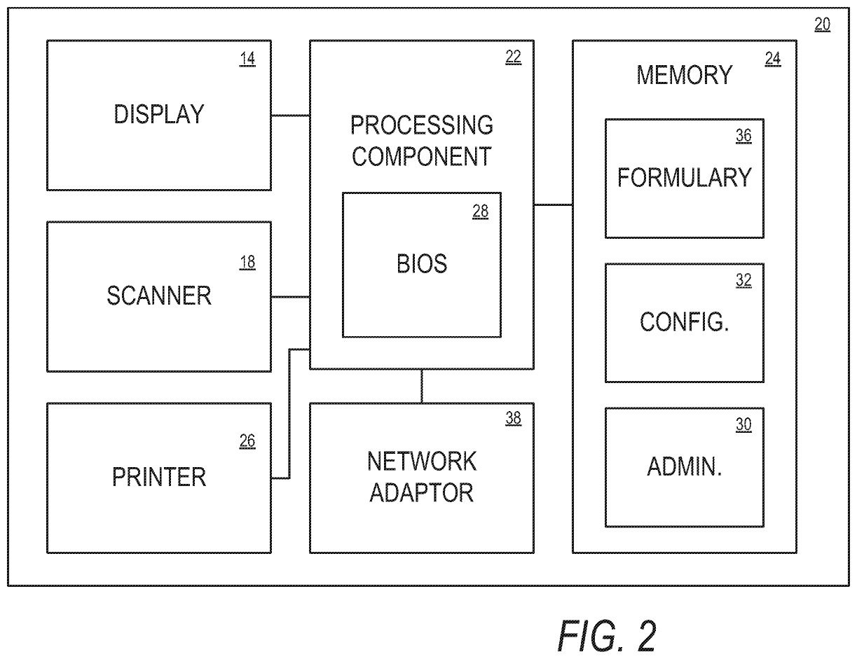

The cabinet 20 also houses or supports components that are operable to produce the label 12 in compliance with a medical labeling standard issued by a medical regulatory agency, professional organization, governing body, government agency, a healthcare provider or facility such as a hospital, or any other standard-setting body that establishes policies for labeling drugs in delivery containers. The internal components housed within the cabinet 20 are schematically illustrated by the block diagram of FIG. 2. The components can be formed from an arrangement of computer hardware such as ASICs, computer processors, programmable logic controllers and other circuitry; or a combination of computer hardware and computer-executable instructions stored in a non-transitory memory 24. For example, a processing component 22 is provided to execute computer-executable instructions stored in a non-transitory, computer-readable memory 24 such as a hard disk drive, read-only memory (“ROM”), random access memory (“RAM”), optical disc, or any other suitable memory device, or any combination thereof. The computer-executed instructions, when executed by the computer processor 22, result in the performance of the method of generating a label for a drug described in detail below. A BIOS 28 is provided to load the operating system and other such administrative instructions 30 stored in the memory 24 and manage hardware interface permissions of the labeling terminal 10. The operating system can be configured to only load authorized updates to prevent unauthorized changes to the formulary 36, configuration data 32 and administration instructions 30. Configuration data 32 controls various features of the labeling terminal 10 that are active and available for use at any given time, including the ability to communicate with an external printer 50 (FIG. 3). The configuration data 32 can optionally be stored, updated and deleted from the memory 24 by the introduction of a so-called smart drive comprising a USB compatible flash memory to the labeling terminal 10. When the smart drive is introduced to the labeling terminal 10, it establishes the configuration data 32 of the labeling terminal 10. The configuration data 32 can optionally be used to deactivate functional features that the labeling terminal 10 would otherwise be able to perform based on the model of the labeling terminal 10. Accordingly, a common hardware platform of the labeling terminal 10 can be configured in a plurality of different functional configurations based on the configuration data 32.

In addition to the administrative instructions 30, the memory 24 also stores an updatable formulary 36 containing a database of medicinal substances that can be identified by the labeling terminal 10 and select information for each medicinal-substance entry in the database. The formulary 36 can optionally be stored, updated and deleted from the memory 24 by the introduction of a so-called smart drive comprising a USB compatible flash memory to the labeling terminal 10. When the smart drive is introduced to the labeling terminal 10, it establishes the formulary 36 of the labeling terminal 10. Illustrative examples of the select information that can be provided for each drug entry includes, but is not limited to, an ID number such as a National Drug Code (“NDC”), UPC code, EAN code, or any other identifying data that can be used to relate a barcode or other computer-readable code to the medicinal-substance entries; a sound file that, when played, audibly announces the name of the medicinal substance identified in response to scanning a machine readable code; warning data; or any combination thereof.

Embodiments of the formulary 36 can also optionally include quantity data associated with one, a plurality or each of the drugs in the formulary 36. The inventory drugs having a field indicative of the number of single use vials, for example, remaining in a drug cart 56 associated with the labeling terminal 10, as shown in FIG. 3, can optionally be monitored by the labeling terminal 10 and/or a remote terminal such as a pharmacy terminal described below, for example, to ensure a sufficient supply of those drugs is available from the cart 56. According to one embodiment, drugs to be monitored can be associated with a minimum threshold field that indicates the minimum quantity of single use vials, for example, that must be stored by the cart 56 as a minimum inventory, as established on a case-by-case basis by the health-care facility where the cart 56 is located. The drug inventory can optionally be stored by a drug cart controller 57, which can communicate with the labeling terminal 10 to track drug consumption. Another field can be associated with a number indicating the actual number of single use vials of at least a portion, and optionally each drug present in the cart 56, and the actual quantities can be compared to the minimum inventory quantities.

A network adaptor 38 (FIG. 2) is operatively connected to communicate with the processing component 22 for communicating with a second, external printer 50, such as that illustrated in FIG. 3. The network adaptor 38 can include any suitable communication port such as a USB port, a hardwired, 10Base-T, 100Base-T, or 1000Base-T Ethernet port, a coaxial cable interface, a fiber-optic interface, any format of wireless communication interface such as an antenna compatible with a short-range communication standard such as Bluetooth, any of the 802.11 standards established by the IEEE, or any combination thereof. Embodiments including wireless network adaptors 38 can employ any desired securing protocol such as WEP, WPA and WPA2, for example, and other suitable security protocol. For embodiments including a network adaptor 38 compatible to communicate over a plurality of different network communication channels, both a hard-wired communication portion of the network adaptor 38 and a wireless communication portion of the network adaptor 38 can optionally be concurrently active. Thus, the labeling terminal 10 can optionally communicate via both the hard-wired and wireless portions of the network adaptor 38 concurrently.

As shown in FIG. 3, the external printer 50 is in communication with the labeling terminal 10 via a USB cable 58 or other suitable communication channel. The external printer 50 defines a pocket 60 (shown in broken lines) into which a fore end of the syringe 35 provided with a Luer taper 64 (FIG. 4) is inserted. A printhead 68 is positioned adjacent to a location in the pocket 60 where the Luer taper 64 is positioned once the forward end of the syringe 35 has been fully inserted into the pocket 60. From this position, the printhead 68 can apply ink or other marking material onto a portion of the Luer taper 64, or a coating or label applied thereto, to print a barcode 72 (FIG. 4) onto the Leur taper 64 of the syringe 35 inserted into the pocket 60 of the external printer 50.

Use of the labeling terminal 10, which includes the printer 26 (optionally an internal printer within the cabinet 20), in combination with the external printer 50 facilitates labeling the syringe 35 in FIG. 4 with one or both of: (i) the label 12 that extends at least partially around the exterior periphery of the barrel region 37 of the syringe 35, and (ii) the barcode 72 applied to the Leur taper 64.

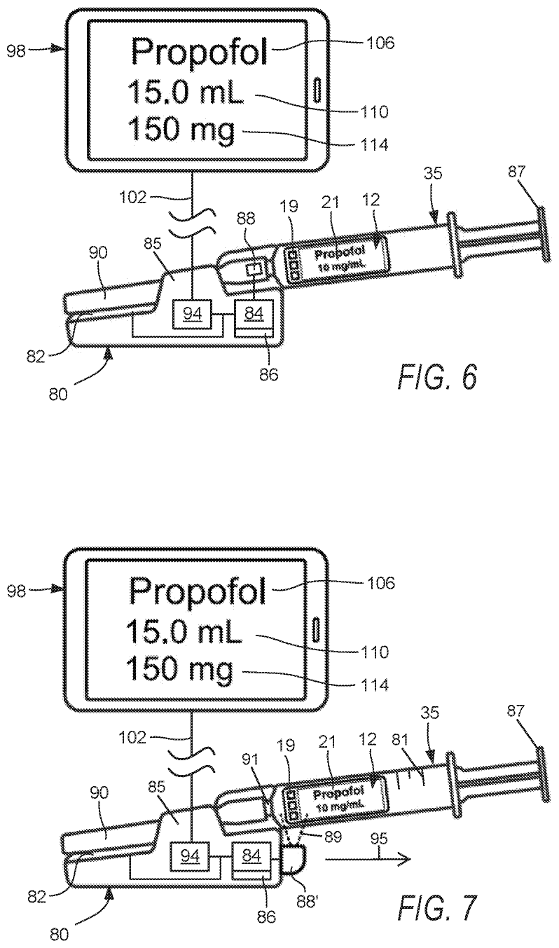

The Luer taper 64 is a standardized type of connection system that forms substantially leak-free connections between a male taper fitting and its mating female part on a needle or infusion line 90 (FIG. 6) such as an IV (intravenous) line, for example, that is connectable to the syringe 35 for delivery of the drug contained in syringe 35 into a patient's body. The Luer taper 64 can be a locking variety, which includes threading that cooperates with a compatible feature provided to the needle or infusion line 90 to “lock” the needle to the syringe 35. As another example, the Luer taper 64 can be a so-called slip type connector that relies on a tight friction fit between the male taper fitting and the mating female part. Regardless of the type of Luer taper, the Luer taper can comply with ISO 80369.

FIG. 5 schematically shows an illustrative embodiment of components included as part of the external printer 50 in communication with the labeling terminal 10. The illustrated embodiment of the external printer 50 includes the aforementioned printhead 68, which applies ink in the appropriate pattern to the Luer taper 64 to form the barcode 72. An ink supply 74 includes at least one, and optionally a plurality or reservoirs of ink available to be emitted by the printhead 68. Optionally, the printhead 68 can use thermal technology that requires no ink to create marks. The printhead 68 can directly apply heat to a material that is thermally activated to change the color of the material. For example, a material can be applied around the Luer taper 64 of syringe 35 (FIG. 4) that is normally white in color, but in the presence of sufficient heat from the printhead 68 the material permanently changes color to gray or black. By controlling the thermal output of printhead 68, a pattern of white and dark regions can be marked on thermally activated material that form the barcode 72 on the Luer taper 64. A memory 78 can store computer-executable instructions that, when executed by a computer processor 82, cause the printhead 68 to print the barcode 72 according to one or more defined barcode symbologies.

Similar to the printer 26 above, the printer 50 can optionally be configured with ink in the ink supply 74 for printing the lines, squares, pixels or other symbols in a color having a contrast that is at least ten (10%) percent lower than the contrast of such lines, squares, pixels or other symbols printed to be black relative to a white background. According to alternate embodiments, the lines, squares, pixels or other symbols can optionally be printed in a color having a contrast that is at least fifteen (15%) percent, or at least twenty (20%) percent lower than the contrast of such lines, squares, pixels or other symbols printed to be black relative to a white background.

The memory 78 can be any memory device that can be accessed by the processor 82, such as a hard disk drive, ROM, RAM, optical disc, or any other suitable memory device, or any combination thereof. The computer-executed instructions, when executed by the computer processor 22, result in the performance of the method of generating and applying the barcode 72 to the Luer taper 64 described in detail below. Further, the memory 78 can store an identifier to be assigned to the syringe 35, drug, preparation, etc. and associated with that is to be encoded by the barcode 72 to be provided to the Luer taper 64.

The barcode 72 applied to the Luer taper 64 is readable when the syringe 35 is installed on a syringe reader 80, shown in FIG. 6. The present embodiment of the syringe reader 80 includes a receiver 85 such as a socket, clasp, cradle (e.g., a recess in which a portion of the syringe 35 is inserted), etc. that cooperates with a portion of the syringe 35 to releasably couple the syringe 35 to the syringe reader 80. Cooperation between the releasable fastener of the receiver 85 and the Luer taper 64 can also establish fluid communication between the syringe 35 and the syringe reader 80. Releasably coupling the syringe 35 to the syringe reader 80 secures the syringe 35 to the syringe reader 80 during the administration of the drug in the syringe 35, yet allows the syringe 35 to be removed and replaced with a different syringe after drug administration is complete. A friction fit, threaded adaptor, or other releasable and reversible fastener that allows the syringe 35 to be removed from the syringe reader 80 after drug administration can be utilized to releasably couple the syringe 35 to the syringe reader 80. A sensor 82 can emit ultrasonic radio waves (or any other wireless signal) can be provided to the syringe reader 80 to sense the flow of the liquid drug passing by the sensor 82 during administration of the drug from the syringe 35. According to other embodiments, a parastaltic sensor or other suitable flow sensor (e.g., wireless signal, capacitive, optical, mechanical, and other types of sensors) can be used to measure the drug volume flowing past the sensor 82 while the plunger 87 of the syringe 35 is manually pressed by a clinician. A signal indicative of the quantity or dose of the drug administered is transmitted by the sensor 82 to be received by a processor 84, which executes instructions stored in a non-transitory memory 86 operatively connected to the processor 84 to determine the volume, dose, or both the volume and dose of the drug administered. Although the processor 84 and memory 86 are show in FIG. 6 as being installed in the syringe reader 80 itself, the processor 84, the memory 86, or both the processor 84 and memory 86 can be located remotely from the housing of the syringe reader 80. For example, the syringe reader 80 can include a cable with a connector compatible with a separate computer terminal, such as the labeling terminal 10, or another remotely located computer terminal. Although the example above describes the manual insertion of the plunger 87 as a biasing force that expels the drug from the syringe 35, other embodiments can optionally utilize gravity as a biasing force acting on an intravenous drip bag, for example.

When the syringe 35 including the barcode 72 on the Luer taper 64 is installed on the syringe reader 80, a barcode scanner 88 positioned on the syringe reader 80 adjacent to the connection between the Luer taper 64 and the infusion line 90 leading to the patient reads the barcode 72. The barcode 72 can optionally be applied to the Luer taper 64 in a manner that allows the barcode scanner 88 to automatically read the barcode 72 during installation of the syringe 35, regardless of the orientation of the syringe 35. For example, the barcode 72 can be printed at one location on the Luer taper 64, and the optics of the barcode scanner 88 can be configured to read the barcode 72 oriented in a plurality of different orientations. According to alternate embodiments, the barcode 72 can be printed to extend a substantial portion about the exterior periphery of the Luer taper 64 such that the barcode scanner 88 can read the barcode 72 regardless of the orientation of the Luer taper 64. Yet other embodiments involve printing a plurality of discrete copies of the barcode 72 at different radial positions about the exterior periphery of the Luer taper 64, ensuring that at least one of the barcodes 72 is readable by a barcode scanner 88 having a limited viewing area at a fixed location on the syringe reader 80. Regardless of the configuration of the barcode 72 and/or the barcode scanner 88, the barcode scanner 88 transmits a signal indicative of the barcode 72 that is read to be received by the processor 84. In response, the processor 84 uses at least a portion of this received information to access a database in the memory 86 to identify the drug in the syringe 35 as identified by the barcode 72.

The syringe reader 80 also includes a transmitter 94 in communication with the processor 84. The transmitter 94 can be compliant with a wireless communication standard such as Bluetooth, any of the IEEE 802.11 standards, a proprietary standard, or any other standard for transmitting information to a display 98 over a wireless communication channel 102. Information transmitted by the transmitter 94 can include at least one of: an identity of the drug identified by the barcode 72, the sensed volume, dose or volume and dose of the drug administered, and any other information about the drug based on the barcode 72 such as an expiration date and/or time, a warning concerning administration of the drug to the patient, and the like. Additionally, the transmitter 94 can send information configured as part of syringe reader 80 that uniquely identifies the particular syringe reader 80 transmitting the information. According to the present embodiment, the label 12 including the human-readable content 21 and the barcode 19 is not used by the syringe reader 80. By processing the signal transmitted by the transmitter 94, the display 98 (e.g., a tablet computer, notebook computer, peripheral computer monitor connected to a desktop computer, etc.) can display information pertaining to the administration of the drug in real time, as the drug is being administered. In FIG. 6, the information presented by the display 98 includes the drug name, total volume administered 110 and the total dose administered 114.

FIG. 7 shows an alternate embodiment of the display 98 in communication with the syringe reader 80 that measures a volume, dose, etc. of a drug actually administered to the patient from the syringe 35 in real time. Instead of, or in addition to the barcode scanner 88, the present embodiment of the syringe reader 80 includes a barcode scanner 88′ positioned to read one or a plurality of barcodes 19 printed by the printer 26 of the labeling terminal 10 onto the label 12 applied to the barrel region 37 of the syringe 35 while the syringe 35 is installed on the syringe reader 80. Such barcodes 19 can be positioned on a portion of the barrel 37 that extends away from the receiver (e.g., protrudes from the cradle or other portion of the receiver). As shown in FIG. 7, the barcode scanner 88′ can include an optics system arranged rearward of the Leur taper 64 of the syringe 35 installed on the syringe reader 80. Thus, the barcode scanner 88′ can have a fixed position relative to the receiver 85, and can be separated from where the Luer taper 64 is to be located while the syringe 35 is installed on the syringe reader 80. At this location, the barcode scanner 88′ is unable to read a barcode applied to the Leur taper 64 of a syringe 35 installed on the syringe reader 80 according to some embodiments. According to alternate embodiments, the optics system can be arranged rearward of the receiver 85 that couples the syringe 35 to the syringe reader 80. The “rearward” direction in FIG. 7 is indicated generally by arrow 95, and extends in a direction generally away from the Leur taper 64 of the syringe installed on the syringe reader 80, or the receiver 85, or an outlet 91 through which the drug is to be expelled from the syringe 35, and toward the plunger 87 of the syringe 35 installed on the syringe reader 80. In other words, the optics system of the barcode reader 88′ is positioned along the direction indicated by the arrow 95 between the Luer taper 64 of the syringe 35 installed on the syringe reader 80 and the plunger 87. The optics system emits a signal 89 that interrogates the barcode(s) 19 applied to the barrel 37 of the syringe 35 to identify one, a plurality, or all of the drug identity, drug concentration, expiration date, a potential allergen to which the patient could exhibit an allergic reaction, a total dose and/or a total volume of the drug to be administered, etc.

A barcode or other computer-readable code such as the barcode 72 applied to the Luer taper 64 of the syringe 35 may not be readable without rotating the syringe 35. The diameter of the Luer taper 64 is generally smaller than the diameter of the barrel region 37, and there is a limited area where the barcode 72 can be applied to the Luer taper 64. To ensure the barcode 72 on the Luer taper 64 is read by the barcode scanner 88 provided to the syringe reader 80 in FIG. 6, the syringe 35 must be positioned with a specific angular orientation in the syringe reader 80 to expose the small barcode 72 to the barcode scanner 88. Doing so may cause graduations 81 (FIG. 7) provided to the barrel region 37 of the syringe 35 to indicate quantities of the drug in the syringe 35 to be facing downward, generally toward a surface that the syringe reader 80 is resting on. Oriented in this manner, the graduations 81 are difficult to read while the syringe 35 is received by the syringe reader 80. Utilizing the barcode scanner 88′ to read the one or more barcodes 19 of the label 12 adhered to the barrel region 37 affords greater flexibility for the orientation of the syringe 35 while it is installed on the syringe reader 80, yet ensuring the barcode 19 is readable by the barcode scanner 88′. Further, utilizing the barcode(s) 19 applied to the label 12 for the barrel region 37 eliminates the need to separately apply the barcode 72 on the Luer taper 64. Otherwise, the syringe reader 80 is the same as the embodiment described above with reference to FIG. 6.

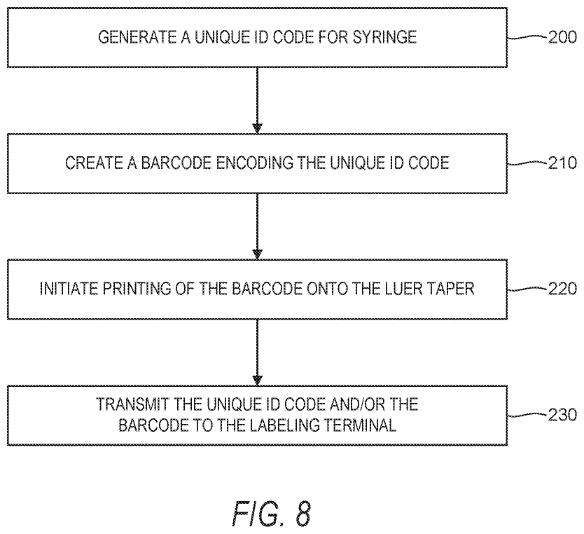

A method of preparing the syringe 35 can be understood with reference to the flow diagram of FIG. 8. The fore end of the syringe 35 provided with a Luer taper 64 is received in the pocket 60 of the external printer 50. Preparing and printing the label content to be applied to the label 12 for the barrel region 37 of the syringe 35 can be done concurrently while a clinician draws the drug from the vial 39 into the syringe 35 and otherwise prepares the syringe 35 according to an efficient workflow. However, the drug cannot be drawn into the syringe 35 while the syringe 35 remains in the pocket 60 of the external printer 50. If the syringe 35 remains unavailable in the pocket 60 for an extended period of time, there will be a reluctance by clinicians to use the external printer 50 or, if such use is mandated, the time required to appropriately prepare each syringe 35 for a given procedure will become prohibitively long for use in the healthcare setting.

To implement an efficient workflow utilizing the external printer 50, the syringe 35 can be assigned a unique identification code at step 200 in FIG. 8. According to an embodiment, the controller 82 (FIG. 5) of the external printer 50 can execute instructions in the memory 78 to generate the unique identification code and prepare the barcode 72 corresponding to the unique identification code to be applied to the Luer taper 64. For example, the controller 82 can utilize time stamp data from an onboard clock to ensure that the unique identification code assigned to the syringe 35 is truly unique, and is not assigned to any other syringe. As another example, the controller 82 can simply increment the numerical value of the unique identification code every time the unique identification code is assigned to a syringe. The unique identification code can optionally be unique to the entire healthcare facility, or at least the location of a facility included in a health system, thus requiring transmission of the unique identification code over a communication network at the facility to ensure no other syringe 35 is assigned the same unique identification code.

Regardless of how the unique code is generated, the unique code is encoded as the barcode 72 at step 210. The controller 82 executes instructions in the memory 78 that cause the controller 82 to initiate operation of the printhead 68, at step 220, to apply the barcode 72 onto the Luer taper 64. Printing of the barcode 72 at step 220 can optionally be initiated before all of the information required to print the label 12 has been received by the labeling terminal 10 to which the external printer 50 is connected. According to alternate embodiments, printing the barcode at step 220 can optionally be initiated such that printing the barcode 220 occurs concurrently with the input of information required to print the label 12 to the labeling terminal 10. According to yet other embodiments, printing the barcode at step 220 can optionally be initiated and completed before the entry of information required to print the label 12 into the labeling terminal 10 even begins. But regardless of when printing of the barcode 72 onto the Luer taper 64 is initiated, printing of that barcode 72 onto the Luer taper 64 is completed before the label 12 with all the appropriate label content becomes available to be applied to the syringe 35. Applying the barcode 72 to the Luer taper 64 in this manner allows the syringe 35 to be removed from the pocket 60 of the external printer 50 so the clinician can begin the process of drawing the drug into the syringe 35 without having to wait for completion of the process for publishing the label 12. Thus, preparing the syringe 35 to include the drug can occur concurrently with the process of receiving, preparing and printing the content for the label 12 performed by the labeling terminal 10.

Because the unique identification code is generated at step 200 by the external printer 50, the external printer 50 transmits information indicative of the unique identification code and/or the barcode 72 to the labeling terminal 10 at step 230 to be stored in the memory 24 of the labeling terminal 10. The processing component 22 of the labeling terminal 10 executes instructions from the memory 24 to create a relationship between the received unique identification code and/or barcode 72 and the barcode 19 that is to be printed onto the label 12 by the printer 26. For example, the barcode 19 provided to the label 12 can be the same as the barcode 72 printed onto the Luer taper 64. The labeling terminal 10 can generate an entry in a database stored in the memory 24 or in a remote storage device for the received unique identification code and/or barcode 72 that includes various information concerning the drug to be administered using the syringe 35. For example, the labeling terminal 10 can store at least one of: the name of the drug, the concentration of the drug, the total dose of the drug to be drawn into the syringe 35, the total volume of the drug to be drawn into the syringe 35, the expiration date and time of the drug, the preparation date and time of the drug, the individual preparing the drug, allergy information indicating the patient is allergic to the drug, warning information about a risk associated with the drug, information identifying the person preparing the syringe 35, the date on which the syringe 35 is prepared, and any other information pertinent to the administration of the drug to the patient.

At least a portion, and optionally all or less than all of the information to be included in the database entry for the unique identification code and/or barcode 72 can optionally be received by the labeling terminal 10 in response to scanning the barcode 41 (FIG. 3) applied to the vial 39. For instance, the barcode 41 can encode the NDC that uniquely identifies the identity of the drug in accordance with US regulations. The labeling terminal 10 can retrieve from the formulary 36 information such as the identity of the drug, drug concentration, etc., to be included in the entry associated with the unique identification code and/or the barcode 72. At least a portion, and optionally all or less than all of the information to be included in the database entry for the unique identification code and/or barcode 72 can optionally be received by the labeling terminal 10 in response to manual user entry of such information. For example, the clinician can select or otherwise input the identity of the drug, the clinician's name, etc. using soft keys displayed by the touch-sensitive display 14 of the labeling terminal 10. The labeling terminal 10 prints the label 12 to include the barcode 19 to encode at least some of the information available to the labeling terminal 10 and includes a portion of that information as human-readable content on the label 12. However, the syringe 35 bearing the barcode 72 on the Luer taper 64 is available to the clinician for drawing the drug from the vial 39 while at least a portion of the process of creating the database entry to include at least a portion of the above information, preparing the label content and printing the label 12 is underway. The labeling terminal 10 can print the label 12, making the completed label 12 available for application to the barrel region 37 of the syringe 35 within ten (10 sec.) seconds, or optionally within seven (7 sec.) seconds, from a time when all the information required to generate the content of the label 12 becomes available to the labeling terminal 10. According to alternate embodiments, the labeling terminal 10 can print the label 12, making the completed label 12 available for application to the barrel region 37 of the syringe 35 within ten (10 sec.) seconds, or optionally within seven (7 sec.) seconds, from a time when the barcode 41 is scanned using the scanner 18 provided to the labeling terminal 10.

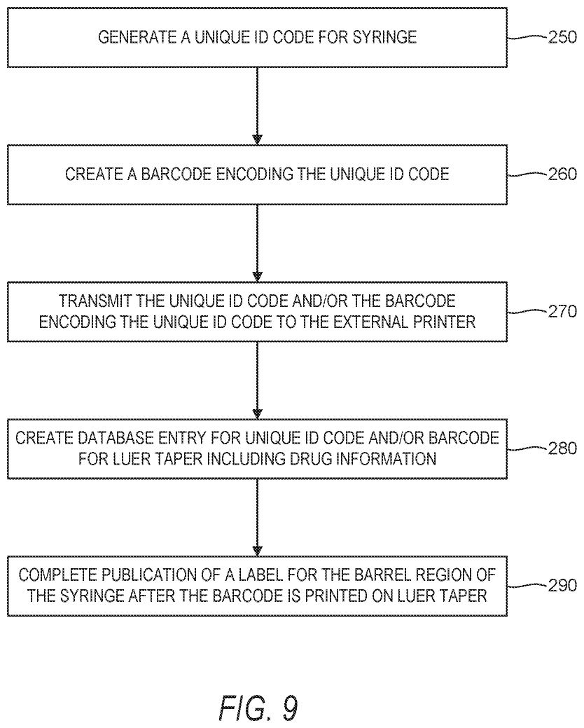

The embodiments described above with reference to FIG. 8 involve the external printer 50 generating the unique identification code and transmitting the unique identification code and/or the barcode 72 to the labeling terminal 10. According to alternate embodiments, the unique identification code can be generated by the labeling terminal 10 and transmitted, optionally as the barcode 72, to the external printer 10. The process according to such an embodiment, graphically illustrated in FIG. 9, involves the processing component 22 of the labeling terminal 10 generating the unique identification code at step 250. The barcode 72 used to encode the unique identification code can also optionally be generated by the computer terminal 10 at step 260. The unique identification code and/or the barcode 72 are transmitted at step 270 to the external printer 50. According to alternate embodiments, the controller 82 of the external printer 50 can optionally generate the barcode 72 based on instructions stored in the memory 78 and the unique identification code received from the labeling terminal 10 according to alternate embodiments. Regardless of the device that generates the barcode 72, the controller 82 of the external printer initiates printing of the barcode 72 encoding the unique identification code generated by the labeling terminal 10 onto the Luer taper 64 in response to introduction of the fore end of the syringe 35 into the pocket 60 of the external printer 50.

Before, after or during transmission of the unique identification code to the external printer, the labeling terminal 10 creates a database entry to include the unique identification code and/or barcode 72 related to information about the drug to be drawn into the syringe 35 at step 280, and proceeds with the process of preparing label content and printing that label content to publish the label 12 at step 290. For example, the scanner 18 can be utilized to read a barcode 41 applied to a vial 39 placed by the clinician under the display 14 of the labeling terminal 10. Subsequent to, and optionally in response to reading this barcode 41, the labeling terminal 10 can transmit the unique identification code and/or the barcode 72 to be applied to the Luer taper 64 to the external printer 50, thus causing the external printer 50 to print the barcode 72 onto the Luer taper 64. Concurrently with, or subsequent to the external printer 50 printing the barcode 72 onto the Luer taper 64, the processing component 22 of the labeling terminal 10 creates a database entry for the unique identification code and/or barcode 72, generates the label content that is to appear on the label 12 and initiates printing the label 12.

The information included in the database entry created by the labeling terminal 10 can include any information about the drug linked to the unique identification code and/or the barcode 72. For instance, the labeling terminal 10 can retrieve from the formulary 36 information such as the identity of the drug, drug concentration, etc., to be included in the entry and associated with the unique identification code and/or the barcode 72. At least a portion, and optionally all or less than all of the information to be included in the database entry for the unique identification code and/or barcode 72 can optionally be received by the labeling terminal 10 in response to manual user entry of such information. For example, the clinician can select or otherwise input the identity of the drug, the clinician's name, etc. using soft keys displayed by the touch-sensitive display 14 of the labeling terminal 10. The labeling terminal 10 prints the label 12 to include the barcode 19 to encode at least some of the information available to the labeling terminal 10 and includes a portion of that information as human-readable content on the label 12. However, the syringe 35 bearing the barcode 72 on the Luer taper 64 is available to the clinician for drawing the drug from the vial 39 while at least a portion of the process of creating the database entry to include at least a portion of the above information, preparing the label content and printing the label 12 is underway. Again, the labeling terminal 10 can print the label 12, making the completed label 12 available for application to the barrel region 37 of the syringe 35 within ten (10 sec.) seconds, or optionally within eight (8 sec.) seconds, from a time when all the information required to generate the content of the label 12 becomes available to the labeling terminal 10. According to alternate embodiments, the labeling terminal 10 can print the label 12, making the completed label 12 available for application to the barrel region 37 of the syringe 35 within ten (10 sec.) seconds, or optionally within eight (8 sec.) seconds, from a time when the barcode 41 is scanned using the scanner 18 provided to the labeling terminal 10.

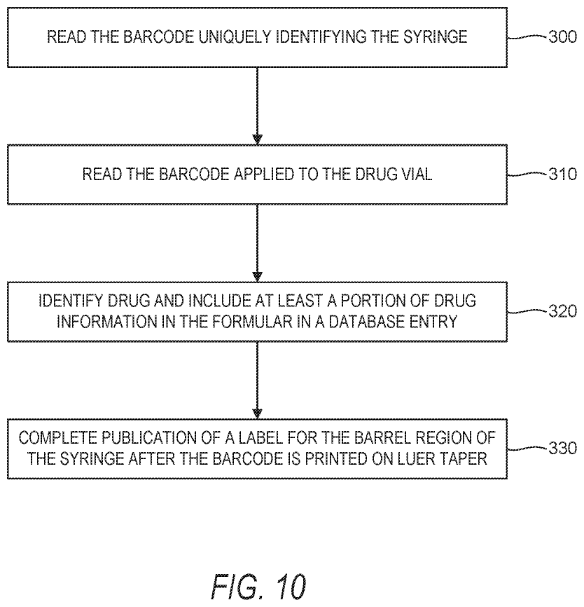

Instead of printing the barcode 72 onto the Luer taper 64, other embodiments utilize a syringe 35 having the barcode 72 printed onto the Luer taper 64 by the syringe manufacturer. In other words, the barcode 72 is printed on the Luer taper 64 before the process of preparing the syringe 35 with the drug begins. According to such embodiments, the external printer 50 can optionally not be used. Instead, as shown in the flow diagram of FIG. 10, the scanner 18 of the labeling terminal 10 can be utilized to read the barcode 72 that was printed on the syringe 35 before the syringe 35 arrived at the healthcare facility at step 300. The processing component 22 (FIG. 2) of the labeling terminal 10 creates a database entry specific to this pre-printed barcode 72. Scanning the barcode 41 (FIG. 3) applied to the drug vial 39 with the scanner 18 at step 310 causes the processing component 22 to identify the drug based on content included in the formulary 36, and optionally retrieve a portion of the drug information in the formulary 36 for the identified drug for inclusion in the database entry at step 320. A portion of the drug information retrieved from the formulary 36 is encoded into the barcode 19 (FIG. 4) that is to appear on the label 12 by the processing component 22, which causes the printer 26 of the labeling terminal 10 to print the label 12 with the barcode 19 at step 330. The barcode 19 printed at step 330 can optionally be the same as the pre-printed barcode 72, or at least encodes information that uniquely identifies the syringe 35 based on the pre-printed barcode 72. Again, the labeling terminal 10 can print the label 12 and make the completed label 12 available for application to the barrel region 37 of the syringe 35 within ten (10 sec.) seconds, or optionally within seven (7 sec.) seconds, from a time when all the information required to generate the content of the label 12 becomes available to the labeling terminal 10. According to alternate embodiments, the labeling terminal 10 can print the label 12 and make the completed label 12 available for application to the barrel region 37 of the syringe 35 within ten (10 sec.) seconds, or optionally within seven (7 sec.) seconds, from a time when the barcode 41 is scanned using the scanner 18 provided to the labeling terminal 10.

Alternately, step 300 shown in the flow diagram of FIG. 10, and step 310 can be reversed such that the drug vial 39 is scanned into terminal 10 first and the barcode 72 that was printed on the syringe 35 before the syringe 35 arrived at the healthcare facility at step 300 is scanned in second. Regardless of the order that step 300 and step 310 are performed, the remainder of the steps in FIG. 10 as previously described are processed the same producing the same completed label 12.

In use, the syringe 35 can be installed on the syringe reader 80 by introducing a compatible portion of the syringe 35 to the receiver 85. The receiver 85 maintains the syringe 35 in place with an orientation to cause at least one, or at least one of a plurality or barcodes 19 printed by the labeling terminal 10 to be read by the barcode scanner 88′. The barcode(s) 19 can be read during installation of the syringe 35 on the syringe reader 80, and/or once the syringe 35 has been completely installed on the syringe reader 80. In response to reading the one or more barcodes 19, the processor 84 can access an electronic health record specific to the patient from a database stored by a network-accessible computer-readable medium (e.g., an anesthesia information management system (“AIMS”), an electronic health record system for a health system, etc.) to access information about the patient. The patient information can include the patient's identity (e.g., ID number, name, or any other identifying information), an order for administration of the drug to the patient, known allergy information for the patient, etc. The retrieved patient information can be compared to information encoded by the barcode(s) 19 to identify any potential conflicts such as the patient being allergic to the drug, the drug is not recommended for this particular patient, the drug was not ordered for administration prior to the beginning of the current medical procedure, or the drug is nearing expiration, for example. In the absence of any warnings regarding one or more potential conflicts, or in the event that a clinician inputs an override instruction to proceed with administration of the drug despite a possible conflict, the sensor 82 measures the quantity of the drug administered. The sensor 82 outputs a signal indicative of the measured quantity, and the processor 84 binds the measured quantity to the patient's health record, thereby documenting the quantity of the drug actually administered.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A syringe reader for measuring a quantity of a drug administered from a syringe, the syringe reader comprising:
a receiver comprising a releasable fastener component that cooperates with a compatible portion of the syringe to releasably couple the syringe to the syringe reader, and maintain cooperation between the syringe and the syringe reader during administration of the drug;
a sensor arranged adjacent to an outlet of the syringe to sense a flow of the drug expelled from the drug container in response to exertion of a biasing force on a plunger of the syringe, and transmit a signal indicative of the sensed flow of the drug; and
a code reader arranged to read a machine-readable code applied to a barrel region of the syringe installed on the syringe reader by the receiver, wherein the code reader comprises a system that emits an interrogation signal to interrogate the machine-readable code applied to the barrel region.

2. The syringe reader of claim 1 further comprising a processor that binds information based on the sensed flow of the drug indicated by the signal transmitted by the sensor to an electronic health record specific to the patient.

3. The syringe reader of claim 2, wherein the processor is programmed to:
access the electronic health record specific to the patient over a communication network, and retrieve allergy information for the patient;
determine that a potential conflict exists by comparing at least a portion of information about the drug obtained as a result of reading the barcode to the allergy information for the patient; and
issuing an alert in response to determining that the potential conflict exists.

4. The syringe reader of claim 1, wherein the code reader is a barcode reader that comprises an optics system that emits an optical signal rearward of the receiver that couples the syringe to the syringe reader.

5. The syringe reader of claim 4, wherein the optics system is positioned away from a location where a Leur taper region of the syringe is positioned while the syringe is installed on the syringe reader.

6. The syringe reader of claim 5, wherein the optics system is unable to emit the optical signal toward a region adjacent to the location where the Leur taper of the syringe is to be positioned while the syringe is installed on the syringe reader.

7. The syringe reader of claim 1, wherein the code reader has a fixed position relative to the receiver, and is positioned to read the machine-readable code while the syringe is installed on the syringe reader.

8. The syringe reader of claim 1, wherein the receiver comprises a recess in which a portion of the syringe is received.

9. The syringe reader of claim 1, wherein the releasable fastener establishes fluid communication between the syringe and the syringe reader.

10. The syringe reader of claim 1, wherein the machine-readable code is a barcode, and the code reader is a barcode reader.

11. The syringe reader of claim 1, wherein the senses a quantity of the drug expelled from the drug container based on the flow.

12. A method of documenting administration of a drug from a syringe coupled to a syringe reader by a receiver comprising a releasable fastener, the method comprising:
with a sensor provided to the syringe reader, sensing a quantity of the drug expelled from the drug container in response to exertion of a biasing force on a plunger of the syringe, and transmitting a signal indicative of the sensed quantity of the drug;
with a barcode reader provided to the syringe reader, reading a barcode applied to a barrel region of the syringe while the syringe is installed on the syringe reader by the receiver, wherein reading the barcode with the barcode reader comprises emitting an optical signal from an optics system having a fixed position relative to the receiver to interrogate the barcode, wherein the fixed position of the optics system is separated from a location where a Luer taper of the syringe installed on the syringe reader is located.

13. The method of claim 12 further comprising:
with a processor programmed with computer-executable instructions, binding the sensed quantity of the drug indicated by the signal transmitted by the sensor to an electronic health record specific to the patient.

14. The method of claim 12 further comprising:
with the processor, accessing an electronic health record specific to the patient over a communication network to retrieve allergy information for the patient;
determining that a potential conflict exists by comparing at least a portion of information about the drug obtained as a result of reading the barcode to the allergy information for the patient retrieved from the electronic health record; and
issuing an alert in response to determining that the potential conflict exists.
